(19) United States
(12) Patent Application Publication
(10) Pub. No.: US 2022/0185569 A1
(43) Pub. Date: Jun. 16, 2022

(54) STORAGE CONTAINER AND A COVER THEREFOR

(71) Applicant: SHENZHEN NANMU ELECTRONIC COMMERCE co., LTD., BANTIAN STREET (CN)

(72) Inventor: WEINAN QI, SHENZHEN (CN)

(21) Appl. No.: 17/117,174

(22) Filed: Dec. 10, 2020

(51) Int. Cl. B65D 81/20; B65D 51/24; B65D 53/02; A61L 2/24; A61L 2/10; A61L 2/20; A61L 2/14

(52) U.S. Cl. CPC B65D 81/2038; B65D 51/248; B65D 53/02; A61L 2/24; A61L 2/10; A61L 2202/14; A61L 2/14; A61L 2202/122; A61L 2202/121; A61L 2202/182; A61L 2202/11; A61L 2/202

(57) ABSTRACT

The present disclosure provides a storage container and a cover for a storage container. The storage container includes a container body having a storage space and an opening communicating with the storage space; and a cover configured for covering the opening of the container body, and the cover includes a vacuum module configured for exhausting air of the storage space and a sterilization module configured for sterilizing the storage space.

### FIELD OF THE INVENTION

The present disclosure relates to storage containers, and more particularly to a storage container and a cover therefor.

### BACKGROUND OF THE INVENTION

Storage containers are used to store articles, mainly used for storing foods or moisture proof articles. With the increasingly higher living level, people enhance their pursuit of quality of life. Traditionally, the storage containers can protect the foods or articles by simply sealing them to a certain extent. However, there exists air in the storage boxes and foods stored for a long time may be prone to oxidize and deteriorate, the articles stored may be damaged in wet weather condition, therefore, the traditional storage containers cannot meet people's increasing living needs.

In order to enhance the food preservation effect and moisture and pest control of articles in the storage containers, the vacuum storage products emerge. However, a vacuum preservation of plastic bags by vacuum machine is not convenient, because it is need to replace the sealed bag after use, and the discarding of plastic bags is not environmentally friendly. In addition, due to a certain amount of air inside a storage container, the preservation time is short. Moreover, due to repeated use, air and water vapor are easy to invade into the storage container, which causes the poor preservation effect and the function of the storage container can not meet the growing diversified needs of life.

### SUMMARY OF THE INVENTION

In order to overcome the disadvantage of the existing storage container, the present disclosure provides a storage container and a cover therefor.

The present disclosure adopts the following technical solution: a storage container, including a container body having a storage space and an opening communicating with the storage space and a cover configured for covering the opening of the container body, the cover including a vacuum module configured for exhausting air of the storage space and a sterilization module configured for sterilizing the storage space.

Further, the sterilization module includes an ultraviolet sterilizer for providing UV light for sterilization.

Further, the ultraviolet sterilizer includes a plurality of ultraviolet lamps for providing the UV light, and the plurality of ultraviolet lamps are arranged in a line.

Further, the sterilization module includes an ozone and negative ion generator for generating ozone and negative ions for sterilization.

Further, the cover includes a humidity and temperature detection element and a display element, the humidity and temperature detection element is configured for detecting humidity and temperature of the storage space, and the display element is configured for display the humidity and temperature of the storage space.

Further, the cover includes a top plate, a bottom plate facing with the top plate and a connection plate connecting the top plate, the bottom plate, the top plate and the connection plate form an accommodate space for accommodating the vacuum module and the sterilization module.

Further, the storage container further includes a seal ring around the bottom plate and located between the cover and the container body for sealing.

Further, the bottom plate includes at least one air hole configured for communicating with the storage space, the vacuum module is disposed on the bottom plate and is connected to the at least one air hole, and the vacuum module exhausts the air of the storage space via the air hole.

Further, the cover includes a control circuit board located in the accommodate space, the at least air hole includes a first hole, the vacuum module includes a vacuum pneumatic pump, an one-way globe valve, a three-way valve and a pressure sensor, the vacuum pneumatic pump is electrically connected to the control circuit board, the one-way globe valve is connected between the vacuum pneumatic pump and a first end of the three-way valve, a second end of the three-way valve is connected to the pressure sensor, a third end of the three-way valve is connected to the first air hole, the pressure sensor is configured for detect a pressure of the storage space, the control circuit board is configured for controlling the vacuum pneumatic pump exhaust air of the storage space based on a pressure detected by the pressure sensor.

Further, the at least air hole includes a second air hole, the vacuum module further includes an electronic valve switch, the electronic valve switch is connected to the second air hole and electrically connected to the control circuit board, the control circuit board is also configured for control the electronic valve switch switch off for sealing the second air hole or switch on for removing the seal of the second air hole.

Further, the cover includes a battery electrically connected to the control circuit board and a charging port configured for charging the battery.

Further, the cover includes at least one control button and an indicator lamp, the control button includes a button portion located at the top plate and a button body located at the control circuit board and corresponding to the button portion, and the indicator lamp includes a lamp cover located at the top plate and a lamp body located at the control circuit board.

The present disclosure also adopts the following technical solution: a cover for a storage container including a vacuum module configured for exhausting air of the storage space; and a sterilization module configured for sterilizing the storage space.

Further, the sterilization module includes an ultraviolet sterilizer for providing UV light for sterilization.

Further, the sterilization module includes an ozone and negative ion generator for generating ozone and negative ions for sterilization.

Further, the cover includes a humidity and temperature detection element and a display element, the humidity and temperature detection element is configured for detecting humidity and temperature of the storage space, and the display element is configured for display the humidity and temperature of the storage space.

Further, the cover includes a top plate, a bottom plate facing with the top plate and a connection plate connecting the top plate, the bottom plate, the top plate and the connection plate form an accommodate space for accommodating the vacuum module and the sterilization module.

Further, the cover includes a control circuit board located in the accommodate space, the at least air hole includes a first hole, the vacuum module includes a vacuum pneumatic pump, an one-way globe valve, a three-way valve and a pressure sensor, the vacuum pneumatic pump is electrically connected to the control circuit board, the one-way globe valve is connected between the vacuum pneumatic pump and a first end of the three-way valve, a second end of the three-way valve is connected to the pressure sensor, a third end of the three-way valve is connected to the first air hole, the pressure sensor is configured for detect a pressure of the storage space, the control circuit board is configured for controlling the vacuum pneumatic pump exhaust air of the storage space based on a pressure detected by the pressure sensor.

Further, the at least air hole includes a second air hole, the vacuum module further includes an electronic valve switch, the electronic valve switch is connected to the second air hole and electrically connected to the control circuit board, the control circuit board is also configured for controlling the electronic valve switch to switch the switch off for sealing the second air hole or switch on for removing the seal of the second air hole.

Further, the cover includes a battery electrically connected to the control circuit board and a charging port configured for charging the battery, the cover includes at least one control button and an indicator lamp, the control button includes a button portion located at the top plate and a button body located at the control circuit board and corresponding to the button portion, and the indicator lamp includes a lamp cover located at the top plate and a lamp body located at the control circuit board.

The present disclosure also has the beneficial effects: the storage container can sterilize the storage space and keep the food, medicine or something else in vacuum for a long time, the storage container is easy to use, the storage container does not need sealing bag and other loss accessories, and is more green and can meet with environmental protection. In addition, the storage container has the ultraviolet sterilization module, the ozone and negative ion generation module, the humidity and temperature detection module and other functional modules according to the different application environment, so as to facilitate the use of different demand environments. Moreover, the storage container does not need other functional electronic vacuum pumping device accessories to use and store at any time, and vacuum storage at any time, and there is no need to search for vacuum pumping equipment everywhere. Furthermore, the storage container can detect the air pressure of the storage space at any time, and can cooperate with the humidity and temperature detection module. When there is air entering the storage space, the vacuum pneumatic pump can be automatically started and vacuum negative pressure sealing can be conducted again, so as to keep the food, medicine or something else in vacuum negative pressure environment for a long time, and the preservation time of the storage container is longer.

### BRIEF DESCRIPTIONS OF THE DRAWINGS

For the purpose of a clearer description of the embodiments in this application or technical solutions in prior art, below is a brief introduction of the attached drawings needed to be used in the description of the embodiments or prior art. Apparently, the attached drawings in the following description are only some embodiments indicated in the present application. For ordinary skill in the art, they may obtain other drawings according to these attached drawings without any innovative laboring.

The present disclosure will be further described with reference to the attached drawings and the embodiments hereunder.

FIG. 1 is a schematic diagram of a storage container according to one embodiment of the present disclosure;

FIG. 2 is an exploded view of the storage container of FIG. 1;

FIG. 3 is a bottom view of the cover of the storage container of FIG. 1; and

FIG. 4 is a cross-section view of the storage container of FIG. 1 taken along line IV-IV of FIG. 1.

### DETAILED DESCRIPTION OF THE ILLUSTRATED EMBODIMENT

In order to provide a clear understanding of the objects, features, and advantages of the embodiments, the following are detailed and complete descriptions to the technological solutions adopted in the embodiments. Obviously, the descriptions are part of the whole embodiments. The other embodiments which are not processed creatively by technicians of ordinary skills in the field are under the protection of this disclosure. The same is given with reference to the drawings and specific embodiments. It should be noted that non-conflicting embodiments in the disclosure and the features in the embodiments may be combined with each other without conflict.

In the following description, numerous specific details are set forth in order to provide a full understanding of the disclosure. The disclosure may be practiced otherwise than as described herein. The following specific embodiments are not to limit the scope of the disclosure.

Unless defined otherwise, all technical and scientific terms herein have the same meaning as used in the field of the art as generally understood. The terms used in the disclosure are to describe particular embodiments and are not intended to limit the disclosure.

The disclosure, referencing the accompanying drawings, is illustrated by way of examples and not by way of limitation. It should be noted that references to “an” or “one” embodiment in this disclosure are not necessarily to the same embodiment, and such references mean “at least one.”

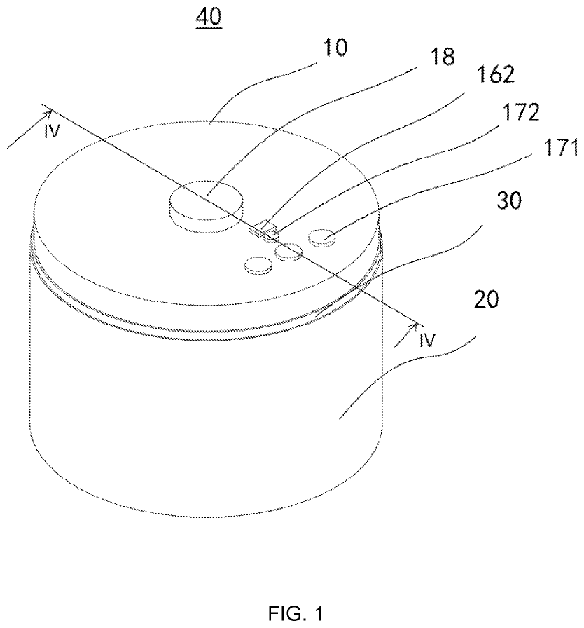

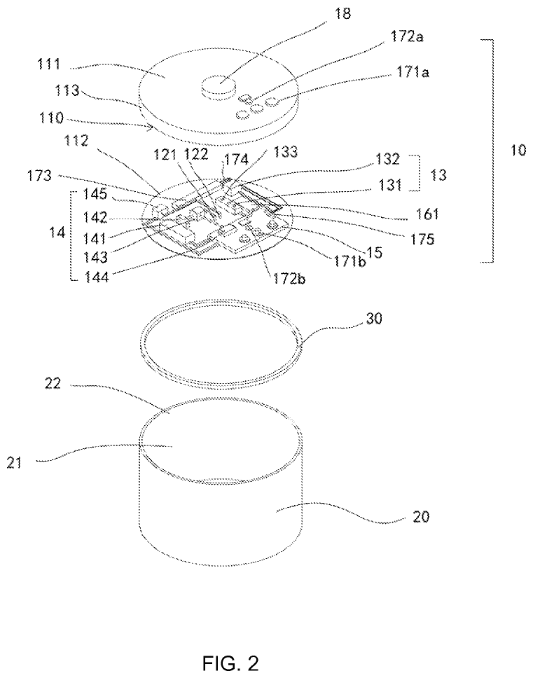

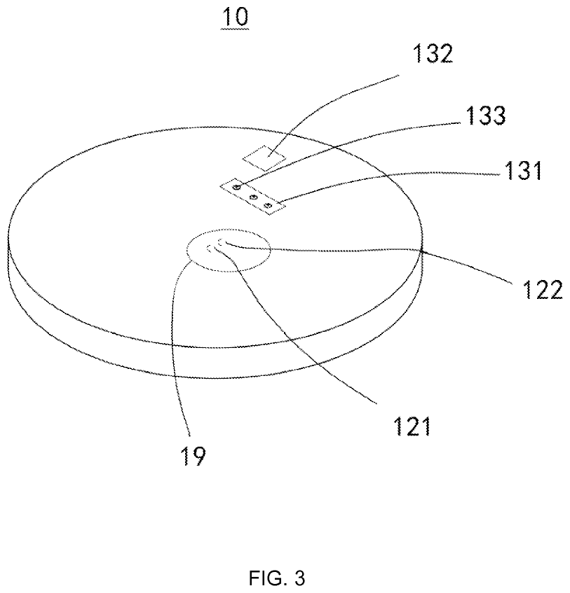

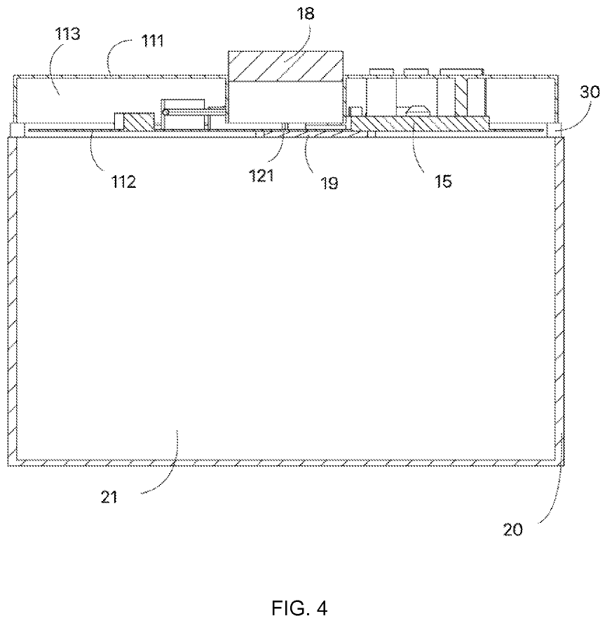

In view of the disadvantage of the existing storage products, the present disclosure provides a storage container. Referring to FIGS. 1-4, the storage container 40 includes a cover 10, a container body 20 and a sealing ring 30. The container body 20 includes a storage space 21 and an opening 22 communicating with the storage space 21.

The storage space 21 is used to storage food, medicine or something else, and it can be made of acrylonitrile butadiene styrene, polycarbonate, silica gel, resin, plastic, rubber, ceramic, glass, stainless steel, metal or a combination of at least two materials of the above materials.

The cover 10 is configured for covering the opening 22 of the container body 20. The cover 10 includes a top plate 111, a bottom plate 112, a connection plate 113, a sterilization module 13, a vacuum module 14, a control circuit board 15, a humidity and temperature detection element 161, a display element 162, a plurality of control buttons 171, an indicator lamp 172, a battery 173, a charging port 175 and a handle 18.

Both of the top plate 111 and the bottom plate 112 can be a round shape and face with each other, and the connection plate 113 connects with an edge of the top plate 111 and extends toward to the bottom plate 112. The bottom plate 112, the top plate 111 and the connection plate 113 form an accommodate space 110 for accommodating at least a part of the sterilization module 13, the vacuum module 14, the control circuit board 15, the humidity and temperature detection element 161, the display element 162, the plurality of control buttons 171, the indicator lamp 172, the battery 173 and the charging port 174. The handle 18 is connected with a side far away from the bottom plate 112 of the top plate 111. The seal ring 30 is located around the bottom plate 112 and between the cover 10 and the container body 20 for sealing. In some embodiments, the seal ring 30 can be integrated with the cover 10 to form an one body element, and in other embodiments, the seal ring 30 can be integrated with the container body 20 to form an one body element.

The sterilization module 13 may include an ultraviolet sterilizer 131 for providing UV light for sterilization and an ozone and negative ion generator 132 for generating ozone and negative ions for sterilization. It can be understood, in some embodiment, the sterilization module 13 may include one of the ultraviolet sterilizer 131 and the ozone and negative ion generator 132. Furthermore, the ultraviolet sterilizer 131 may include a plurality of ultraviolet lamps 133 for providing the UV light, and the plurality of ultraviolet lamps 133 are arranged in a line.

The vacuum module 14 is configured for exhausting air of the storage space 22 when the cover 10 covers on the container body 20 and the sealing ring 30 is located between the cover 10 and the container body 20. The bottom plate 112 includes air holes 121, 122 configured for communicating with the storage space 21, the vacuum module 14 is disposed on the bottom plate 112 and is connected to the air holes 121, 122, and the vacuum module 14 exhausts the air of the storage space 21 via the air holes 121,122.

The vacuum module 14 may include a vacuum pneumatic pump 141, a one-way globe valve 142, a three-way valve 143, a pressure sensor 144 and an electronic valve switch 145. The vacuum pneumatic pump 141 is electrically connected to the control circuit board 15. The one-way globe valve 142 is connected between the vacuum pneumatic pump 141 and a first end of the three-way valve 143, a second end of the three-way valve 143 is connected to the pressure sensor 144, and a third end of the three-way valve 143 is connected to the air hole 121. The pressure sensor 144 is configured 1 for detect a pressure of the storage space 21. The control circuit board 15 is configured for controlling the vacuum pneumatic pump 141 exhaust air of the storage space 21 based on the pressure detected by the pressure sensor 144.

The electronic valve switch 145 is connected to the air hole 122 and electrically connected to the control circuit board 15, the control circuit board 15 is also configured for control the electronic valve switch 145 switch off for sealing the air hole 122 or switch on for removing the seal of the air hole 122. When the seal of the air hole 122 is removed, air can enter into the storage space 21 and the cover 10 can be detached from the container body 11 easily.

In the embodiment, the cover 10 further includes a filter element 19 located at a side far away from the top plate 111 of the bottom plate 112. The filter element 19 is disposed corresponding to the air holes 121,122, and the filter element 19 is configured to filter air before the air enters of the air holes 121, 122 in order to prevent the air holes 121,122 from blocking by the food, medicine or something else.

The humidity and temperature detection element 161 is configured for detecting humidity and temperature of the storage space 21, and the display element 162 is configured for display the humidity and temperature of the storage space 21. The humidity and temperature detection element 161 may output a humidity and temperature signal to the control circuit board 15, and the control circuit board 15 may control the display element 162 display humidity and temperature information via the top plate 11 based on the humidity and temperature signal, such that the humidity and temperature information of the storage space 21 can be provided to users in real time. The display element 162 may be a liquid crystal panel.

The battery 173 is electrically connected to the control circuit board 15 via the connection lines 174. The charging port 175 is configured for charging the battery 173 and located at the control circuit board 15.

The control button 171 may include a button portion 171a located at the top plate 111 and a button body 171b located at the control circuit board 15 and corresponding to the button portion 171a. The indicator lamp 172 includes a lamp cover 172a located at the top plate 111 and a lamp body 172b located at the control circuit board 15. In other embodiment, the control buttons 171 may be capacitive touch buttons, resistive touch buttons, infrared touch buttons and the like.

When the food, medicine or something else is putted into the storage space 21, a user can cover the cover 10 on the container body 20 with the seal ring 30 located between the cover 10 and the container body 20 for sealing the storage space 21. The control button 171 for exhausting air may be pressed by the user and the control circuit board 15 controls the vacuum pneumatic pump 141 start to exhaust the air of the storage space 21 via the one-way globe valve 142, the three-way valve 143 and the air hole 121. The pressure sensor 144 detects a pressure of the storage space 21 and when the pressure of the storage space 21 is greater than or equal to a predetermined value, the control circuit board 15 controls vacuum pneumatic pump 141 stop to exhaust the air of the storage space 21 and the control circuit board 15 is also configured for control the electronic valve switch 145 switch off for sealing the air hole 122.

After a few hours or days, when little air enters the storage space 21 and the pressure of the storage space 21 is less than the predetermined value, the control circuit board 15 controls vacuum pneumatic pump start to exhaust the air of the storage space 21 until the pressure of the storage space 21 is greater than or equal to the predetermined value.

In addition, the control button 171 for ultraviolet sterilization may be pressed by the user to control the ultraviolet sterilizer 131 provide UV light to the storage space 21, and the control button 171 for ozone and negative ion sterilization may be pressed by the user to control the ozone and negative ion generator 132 provide ozone and negative ions to the storage space 21. The humidity and temperature detection element 161 outputs a humidity and temperature signal to the control circuit board 15, and the control circuit board 15 controls the display element 162 display humidity and temperature information via the top plate 11 based on the humidity and temperature signal, such that the humidity and temperature information of the storage space 21 can be provided to users in real time.

Furthermore, when the control button 171 for removing the vacuum of the storage space 21 is pressed by the user, the control circuit board 15 controls the electronic valve switch 145 switch on for removing the seal of the air hole 122, and then air can enter into the storage space 21 and the cover 10 can be detached from the container body 11 easily.

The present disclosure also has the beneficial effects: the storage container 40 can sterilize the storage space and keep the food, medicine or something else in vacuum for a long time, the storage container 40 is easy to use, the storage container does not need sealing bag and other loss accessories, and is more green and can meet with environmental protection. In addition, the storage container 40 has the ultraviolet sterilization module, the ozone and negative ion generation module, the humidity and temperature detection module and other functional modules according to the different application environment, so as to facilitate the use of different demand environments. Moreover, the storage container 40 does not need other functional electronic vacuum pumping device accessories to use and store at any time, and vacuum storage at any time, and there is no need to search for vacuum pumping equipment everywhere. Furthermore, the storage container 40 can detect the air pressure of the storage space 21 at any time by the pressure sensor 144, and can cooperate with the humidity and temperature detection module 161. When there is air entering the storage space, the vacuum pneumatic pump 141 can be automatically started and vacuum negative pressure sealing can be conducted again, so as to keep the food, medicine or something else in vacuum negative pressure environment for a long time, and the preservation time of the storage container is longer.

Finally, it should be noted that above embodiments are merely used for illustrating the technical solutions of the disclosure, rather than limiting the disclosure; though the disclosure is illustrated in detail with reference to the aforementioned embodiments, it should be understood by those of ordinary skill in the art that modifications may still be made on the technical solutions disclosed in the aforementioned respective embodiments, or equivalent substitutions may be made to a part of technical features thereof; and these modifications or substitutions do not make the essence of the corresponding technical solutions depart from the spirit and scope of the technical solutions of the respective embodiments of the disclosure.

What is claimed as new and desired to be protected by Letters Patent, is:

1. A storage container, comprising:
a container body comprising a storage space and an opening communicating with the storage space; and
a cover configured for covering the opening of the container body, the cover comprising a vacuum module configured for exhausting air of the storage space and a sterilization module configured for sterilizing the storage space.

2. The storage container of claim 1, wherein the sterilization module comprises an ultraviolet sterilizer for providing UV light for sterilization.

3. The storage container of claim 2, wherein the ultraviolet sterilizer comprises a plurality of ultraviolet lamps for providing the UV light, and the plurality of ultraviolet lamps are arranged in a line.

4. The storage container of claim 1, wherein the sterilization module comprises an ozone and negative ion generator for generating ozone and negative ions for sterilization.

5. The storage container of claim 1, wherein the cover further comprises a humidity and temperature detection element and a display element, the humidity and temperature detection element is configured for detecting humidity and temperature of the storage space, and the display element is configured for display the humidity and temperature of the storage space.

6. The storage container of claim 1, wherein the cover comprises a top plate, a bottom plate facing with the top plate and a connection plate connecting the top plate, the bottom plate, the top plate and the connection plate form an accommodate space for accommodating the vacuum module and the sterilization module.

7. The storage container of claim 6, wherein the storage container further comprises a seal ring around the bottom plate and located between the cover and the container body for sealing.

8. The storage container of claim 6, wherein the bottom plate comprises at least one air hole configured for communicating with the storage space, the vacuum module is disposed on the bottom plate and is connected to the at least one air hole, and the vacuum module exhausts the air of the storage space via the air hole.

9. The storage container of claim 8, wherein the cover further comprises a control circuit board located in the accommodate space, the at least air hole comprises a first hole, the vacuum module comprises a vacuum pneumatic pump, an one-way globe valve, a three-way valve and a pressure sensor, the vacuum pneumatic pump is electrically connected to the control circuit board, the one-way globe valve is connected between the vacuum pneumatic pump and a first end of the three-way valve, a second end of the three-way valve is connected to the pressure sensor, a third end of the three-way valve is connected to the first air hole, the pressure sensor is configured for detect a pressure of the storage space, the control circuit board is configured for controlling the vacuum pneumatic pump exhaust air of the storage space based on a pressure detected by the pressure sensor.

10. The storage container of claim 9, wherein the at least air hole comprises a second air hole, the vacuum module further comprises an electronic valve switch, the electronic valve switch is connected to the second air hole and electrically connected to the control circuit board, the control circuit board is also configured for controlling the electronic valve switch to switch off so as to seal the second air hole or switch on for removing the seal of the second air hole.

11. The storage container of claim 9, the cover further comprises a battery electrically connected to the control circuit board and a charging port configured for charging the battery.

12. The storage container of claim 9, the cover further comprises at least one control button and an indicator lamp, the control button comprises a button portion located at the top plate and a button body located at the control circuit board and corresponding to the button portion, and the indicator lamp comprises a lamp cover located at the top plate and a lamp body located at the control circuit board.

13. A cover for a storage container, comprising:
a vacuum module configured for exhausting air of the storage space; and
a sterilization module configured for sterilizing the storage space.

14. The cover of claim 13, wherein the sterilization module comprises an ultraviolet sterilizer for providing UV light for sterilization.

15. The cover of claim 13, wherein the sterilization module comprises an ozone and negative ion generator for generating ozone and negative ions for sterilization.

16. The cover of claim 13, wherein the cover further comprises a humidity and temperature detection element and a display element, the humidity and temperature detection element is configured for detecting humidity and temperature of the storage space, and the display element is configured for display the humidity and temperature of the storage space.

17. The cover of claim 13, wherein the cover comprises a top plate, a bottom plate facing with the top plate and a connection plate connecting the top plate, the bottom plate, the top plate and the connection plate form an accommodate space for accommodating the vacuum module and the sterilization module.

18. The cover of claim 17, wherein the cover further comprises a control circuit board located in the accommodate space, the bottom plate comprises a first air hole configured for communicating with the storage space, the vacuum module comprises a vacuum pneumatic pump, an one-way globe valve, a three-way valve and a pressure sensor, the vacuum pneumatic pump is electrically connected to the control circuit board, the one-way globe valve is connected between the vacuum pneumatic pump and a first end of the three-way valve, a second end of the three-way valve is connected to the pressure sensor, a third end of the three-way valve is connected to the first air hole, the pressure sensor is configured for detect a pressure of the storage space, the control circuit board is configured for controlling the vacuum pneumatic pump exhaust air of the storage space based on a pressure detected by the pressure sensor.

19. The cover of claim 18, wherein the at least air hole comprises a second air hole, the vacuum module further comprises an electronic valve switch, the electronic valve switch is connected to the second air hole and electrically connected to the control circuit board, the control circuit board is also configured for controlling the electronic valve switch to switch off so as to seal the second air hole or switch on for removing the seal of the second air hole.

20. The cover of claim 18, the cover further comprises a battery electrically connected to the control circuit board and a charging port configured for charging the battery; the cover further comprises at least one control button and an indicator lamp, the control button comprises a button portion located at the top plate and a button body located at the control circuit board and corresponding to the button portion, and the indicator lamp comprises a lamp cover located at the top plate and a lamp body located at the control circuit board.
